(19) United States
(12) Patent Application Publication
(10) Pub. No.: US 2005/0136095 A1
(43) Pub. Date: Jun. 23, 2005

(54) Drug delivery device with suture ring

(76) Inventors: Brian Levy, Rochester, NY (US); David Watson, San Jose, CA (US); George Purtell, Westford, MA (US); Dominic V. Ruscio, Webster, NY (US); Jay F. Kunzler, Canandaigua, NY (US); Michael M. Schmidt, Cambridge, MA (US); Matthew S. Jonasse, Sodus, NY (US); Joseph C. Salamone, Fairport, NY (US)

Correspondence Address: Bausch & Lomb Incorporated, One Bausch & Lomb Place, Rochester, NY, 14604-2701, US

(21) Appl. No.: 11/012,806

(22) Filed: Dec. 15, 2004

Related U.S. Application Data
(60) Provisional application No. 60/531,847, filed on Dec. 22, 2003.

(51) Int. Cl. A61K 9/22; A61F 2/00

(52) U.S. Cl. 424/427; 604/890.1

(57) ABSTRACT

A drug delivery device for placement in the eye includes a drug core comprising a pharmaceutically active agent, and a holder that holds the drug core. The holder is made of a material impermeable to passage of the active agent and includes an opening for passage of the pharmaceutically agent therethrough to eye tissue. The holder has a suture tab having a suture ring at an end thereof. The suture ring is used to secure the device at an implant site.

### FIELD OF THE INVENTION

This invention relates to a drug delivery device, preferably a device that is placed or implanted in the eye to release a pharmaceutically active agent to the eye. The device includes a drug core and a holder for the drug core, wherein the holder is made of a material impermeable to passage of the active agent and includes at least one opening for passage of the pharmaceutical agent therethrough to the eye tissue. The device further includes a suture tab having a suture ring for securing the device to, for example, a structure of the eye.

### BACKGROUND OF THE INVENTION

Various drugs have been developed to assist in the treatment of a wide variety of ailments and diseases. However, in many instances, such drugs cannot be effectively administered orally or intravenously without the risk of detrimental side effects. Additionally, it is often desired to administer a drug locally, i.e., to the area of the body requiring treatment. Further, it may be desired to administer a drug locally in a sustained release manner, so that relatively small doses of the drug are exposed to the area of the body requiring treatment over an extended period of time.

Accordingly, various sustained release drug delivery devices have been proposed for placing in the eye for treating various eye diseases. Examples are found in the following patents, the disclosures of which are incorporated herein by reference: US 2002/0086051A1 (Viscasillas); US 2002/0106395A1 (Brubaker); US 2002/0110591A1 (Brubaker et al.); US 2002/0110592A1 (Brubaker et al.); US 2002/0110635A1 (Brubaker et al.); U.S. Pat. No. 5,378,475 (Smith et al.); U.S. Pat. No. 5,773,019 (Ashton et al.); U.S. Pat. No. 5,902,598 (Chen et al.); U.S. Pat. No. 6,001,386 (Ashton et al.); U.S. Pat. No. 6,375,972 (Guo et al.); U.S. patent application Ser. No. 10/403,421 (Drug Delivery Device, filed Mar. 28, 2003) (Mosack et al.); and U.S. patent application Ser. No. 10/610,063 (Drug Delivery Device, filed Jun. 30, 2003) (Mosack).

Many of these devices include an inner drug core having a pharmaceutically active agent, and some type of holder for the drug core made of an impermeable material such as silicone or other hydrophobic materials. The holder includes one or more openings for passage of the pharmaceutically active agent through the impermeable material to eye tissue. Many of these devices include at least one layer of material permeable to the active agent, such as polyvinyl alcohol (PVA).

Attached to or integral with the holder is a suture tab for securing the drug delivery device to the area of the body requiring treatment. The suture tab contains a suture hole at one end of the device. In use, the suture material used by the surgeon to attach the device at the implant site can weaken or tear the suture hole. Therefore, an improved drug delivery device that can be attached to a patient without weakening or damaging the suture tab would be desirable.

### BRIEF DESCRIPTION OF THE DRAWINGS

FIG. 1 is a top view of a first embodiment of a drug delivery device of this invention.

FIG. 2 is a side view of the device of FIG. 1.

FIG. 3 is a side view of a second embodiment of a drug delivery device.

FIG. 4 is a side view of a third embodiment of a drug delivery device.

### SUMMARY OF THE INVENTION

According to a first embodiment, this invention relates to a drug delivery device for placement in the eye, comprising: a drug core comprising a pharmaceutically active agent; and a holder that holds the drug core, the holder being made of a material impermeable to passage of the active agent and including an opening for passage of the pharmaceutically active agent therethrough to eye tissue. Integral with the holder is a suture tab thereby providing a unitary drug holder and suture tab. The suture tab contains a suture hole at one end of the device where a suture ring is secured to facilitate the retention of the device at an implant site. The suture ring minimizes potential failure of the suture tab at the suture hole. The suture ring can be prepared from a variety of high strength, currently available materials, for example, ophthalmic suture materials. The invention further comprises an an assembly for storing the device during storage and shipping. In one embodiment the assembly comprises an upper surface; a first flange extending upwardly from the upper surface and defining a containment region for containing the device, said containment region including a support surface for supporting the device in the containment region; a second flange extending upwardly from the upper surface, said second flange surrounding the first flange and including an upper flange surface for sealing of lidstock thereto; and at least one side wall extending downwardly from the upper surface and serving to support the package on a work surface, further comprising a recess extending below the device support surface in the containment region, wherein the first flange comprises two protrusions extending upwardly from the upper surface and defining the containment region, and the recess has the form of an elongated groove separating the two protrusions and extending transversely to the containment region, wherein the two protrusions are arcuate, wherein the maximum width between inner surfaces of an individual protrusion is 10 mm.

### DETAILED DESCRIPTION OF PREFERRED EMBODIMENTS

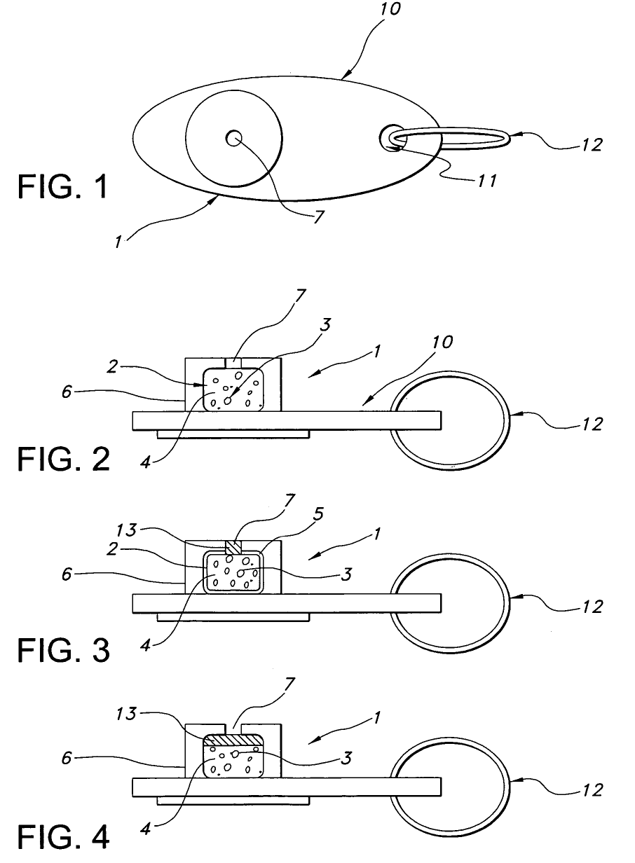

FIGS. 1 and 2 illustrate a first embodiment of a device of this invention. Device 1 is a sustained release drug delivery device for implanting in the eye. Device 1 includes inner drug core 2 including a pharmaceutically active agent 3.

The active agent may include any compound, composition of matter, or mixture thereof that can be delivered from the device to produce a beneficial and useful result to the eye, especially an agent effective in obtaining a desired local or systemic physiological or pharmacological effect. Examples of such agents include: anesthetics and pain killing agents such as lidocaine and related compounds and benzodiazepam and related compounds; benzodiazepine receptor agonists such as abecamil; GABA receptor modulators such as baclofen, muscimol and benzodiazepines; anti-cancer agents such as 5-fluorouracil, adriamycin and related compounds; anti-fungal agents such as fluconazole and related compounds; anti-viral agents such as trisodium phosphomonoformate, trifluorothymidine, acyclovir, ganciclovir, DDI and AZT; cell transport/mobility agents impeding such as colchicine, vincristine, cytochalasin B and related compounds; antiglaucoma drugs such as beta-blockers: timolol, betaxolol, atenalol, etc; antihypertensives; decongestants such as phenylephrine, naphazoline, and tetrahydrazoline; immunological response modifiers such as muramyl dipeptide and related compounds; peptides and proteins such as cyclosporin, insulin, growth hormones, insulin related growth factor, heat shock proteins and related compounds; steroidal compounds such as dexamethasone, prednisolone and related compounds; low solubility steroids such as fluocinolone acetonide and related compounds; carbonic anhydrase inhibitors; diagnostic agents; antiapoptosis agents; gene therapy agents; sequestering agents; reductants such as glutathione; antipermeability agents; antisense compounds; antiproliferative agents; antibody conjugates; antidepressants; bloodflow enhancers; antiasthmatic drugs; antiparasitic agents; non-steroidal antiinflammatory agents such as ibuprofen; nutrients and vitamins: enzyme inhibitors: antioxidants; anticataract drugs; aldose reductase inhibitors; cytoprotectants; cytokines, cytokine inhibitors and cytokine protectants; uv blockers; mast cell stabilizers; and antineovascular agents such as antiangiogenic agents like matrix metalloprotease inhibitors.

Examples of such agents also include: neuroprotectants such as nimodipine and related compounds; antibiotics such as tetracycline, chlortetracycline, bacitracin, neomycin, polymyxin, gramicidin, oxytetracycline, chloramphenicol, gentamycin, and erythromycin; antiinfectives; antibacterials such as sulfonamides, sulfacetamide, sulfamethizole, sulfisoxazole; nitrofurazone, and sodium propionate; antiallergenics such as antazoline, methapyriline, chlorpheniramine, pyrilamine and prophenpyridamine; antiinflammatories such as hydrocortisone, hydrocortisone acetate, dexamethasone 21-phosphate, fluocinolone, medrysone, methyiprednisolone, prednisolone 21-phosphate, prednisolone acetate, fluoromethalone, betamethasone and triminolone; miotics and anti-cholinesterase such as pilocarpine, eseridine salicylate, carbachol, diisopropyl fluorophosphate, phospholine iodine, and demecarium bromide; mydriatics such as atropine sulfate, cyclopentolate, homatropine, scopolamine, tropicamide, eucatropine, and hydroxyamphetamine; sympathomimetics such as epinephrine; and prodrugs such as those described in Design of Prodrugs, edited by Hans Bundgaard, Elsevier Scientific Publishing Co., Amsterdam, 1985. In addition to the above agents, other agents suitable for treating, managing, or diagnosing conditions in a mammalian organism may be placed in the inner core and administered using the sustained release drug delivery devices of the current invention. Once again, reference may be made to any standard pharmaceutical textbook such as Remington's Pharmaceutical Sciences for the identity of other agents.

Any pharmaceutically acceptable form of such a compound may be employed in the practice of the present invention, i.e., the free base or a pharmaceutically acceptable salt or ester thereof. Pharmaceutically acceptable salts, for instance, include sulfate, lactate, acetate, stearate, hydrochloride, tartrate, maleate and the like.

For the illustrated embodiment, the active agent employed is fluocininolone acetonide.

As shown in FIG. 3, active agent 3 may be mixed with a matrix material 4. Preferably, matrix material 4 is a polymeric material that is compatible with body fluids and the eye. Additionally, matrix material 4 should be permeable to passage of the active agent 3 therethrough, particularly when the device 1 is exposed to body fluids. For this embodiment, the matrix material 4 is PVA. Also, in this embodiment, inner drug core 2 may be coated with a coating 5 of additional matrix material which may be the same or different from material 4 mixed with the active agent. For the illustrated embodiment, the coating 5 employed is also PVA. The coating 5 should be a material permeable or semi-permeable to active agent 3.

Device 1 includes a holder 6 for the inner drug core 2. Holder 6 is made of a material that is impermeable to passage of the active agent 3 therethrough. Since holder 6 is made of the impermeable material, at least one passageway 7 is formed in holder 6 to permit active agent 3 to pass therethrough and contact eye tissue. In other words, active agent 3 passes through any permeable matrix material 4 and permeable or semi-permeable coating 5, and exits the device through passageway 7. For the illustrated embodiment, the holder 6 is made of silicone, especially polydimethylsiloxane (PDMS) material.

Materials suitable as coating 5 would include materials that are non-bioerodible and are permeable or can be made to be permeable to the active agent. Preferably, the coating material will be release rate limiting. Suitable polymers, depending upon the specific active agent, would include polyvinyl alcohol, ethylene vinyl acetate, silicone, polylactic acid, nylon, polypropylene, polycarbonate, cellulose, cellulose acetate, polyglycolic acid, polylactic glycolic acid, cellulose esters or polyether sulfone. Coating 5 may also be any of the various semipermeable membrane-forming compositions or polymers such as those described in U.S. patent Publication No. 2002/0197316 (hereby incorporated by reference). Coating 5 may also include plasticizer and pharmaceutically acceptable surfactant such as those described in U.S. patent Publication No. 2002/0197316.

Further examples of semipermeable polymers that may be useful according to the invention herein can be found in U.S. Pat. No. 4,285,987 (hereby incorporated by reference), as well as the selectively permeable polymers formed by the coprecipitation of a polycation and a polyanion as described in U.S. Pat. Nos. 3,541,005; 3,541,006 and 3,546,142 (hereby incorporated by reference).

In addition to the illustrated materials, a wide variety of materials may be used to construct the devices of the present invention. The only requirements are that they are inert; non-immunogenic and of the desired permeability. Materials that may be suitable for fabricating the device 1 include naturally occurring or synthetic materials that are biologically compatible with body fluids and body tissues, and essentially insoluble in the body fluids with which the material will come in contact. The use of rapidly dissolving materials or materials highly soluble in body fluids are to be avoided since dissolution of the wall would affect the constancy of the drug release, as well as the capability of the device 1 to remain in place for a prolonged period of time.

Naturally occurring or synthetic materials that are biologically compatible with body fluids and eye tissues and essentially insoluble in body fluids which the material will come in contact include, but are not limited to, glass, metal, ceramics, polyvinyl acetate, cross-linked polyvinyl alcohol, cross-linked polyvinyl butyrate, ethylene ethylacrylate copolymer, polyethyl hexylacrylate, polyvinyl chloride, polyvinyl acetals, plasiticized ethylene vinylacetate copolymer, polyvinyl alcohol, polyvinyl acetate, ethylene vinylchloride copolymer, polyvinyl esters, polyvinylbutyrate, polyvinylformal, polyamides, polymethylmethacrylate, polybutylmethacrylate, plasticized polyvinyl chloride, plasticized nylon, plasticized soft nylon, plasticized polyethylene terephthalate, natural rubber, polyisoprene, polyisobutylene, polybutadiene, polyethylene, polytetrafluoroethylene, polyvinylidene chloride, polyacrylonitrile, cross-linked polyvinylpyrrolidone, polytrifluorochloroethylene, chlorinated polyethylene, poly(1,4′-isopropylidene diphenylene carbonate), vinylidene chloride, acrylonitrile copolymer, vinyl chloride-diethyl fumerate copolymer, butadiene/styrene copolymers, silicone rubbers, especially the medical grade polydimethylsiloxanes, ethylene-propylene rubber, silicone-carbonate copolymers, vinylidene chloride-vinyl chloride copolymer, vinyl chloride-acrylonitrile copolymer and vinylidene chloride-acrylonitride copolymer.

Device 1 has a suture tab 10 having a suture hole 11 at one end thereof. The tab may be a monolithic aspect of device 1 or it may be adhered to the holder. The suture hole 11 has a suture ring 12 placed therethrough. The suture ring 12 can be prepared from a variety of high strength, biocompatible materials. Examples of suitable materials would include nonabsorbable ophthalmic suture materials such as ETHILON® nylon suture, MERSILENE® polyester fiber suture, PERMA-HAND® silk suture, PROLENE® polypropylene suture, each commercially available from Ethicon, Somerville, N.J.; and VASCUFIL® coated monofilament suture composed of a copolymer of butylene terephthalate and polyteramethylene ether glycol, MONOSOF˜DERMALON® monofilament nylon sutures composed of long-chain aliphatic polymers Nylon 6 and Nylon 6.6, NOVAFIL® monofilament sutures composed of a copolymer of butylene terephthalate and polyteramethylene ether glycol, SOFSILK® braided sutures composed of fibroin, TI-CRON-SURGIDAC® braided polyester sutures composed of polyester terephthalate, SURGILON® braided nylon sutures composed of the long-chain aliphatic polymers Nylon 6 and Nylon 6.6 and SURGIPRO II˜SURGIPRO® sutures composed of polypropylene, each commercially available from U.S. Surgical, Norwalk, Conn. Materials that may be suitable for fabricating the suture ring of the device include naturally occurring or synthetic materials that are biologically compatible with body fluids and body tissues, and essentially insoluble in the body fluids with which the material will come in contact. The use of rapidly dissolving materials or materials highly soluble in body fluids are to be avoided since dissolution of the suture ring would affect the capability of the device to remain in place for a prolonged period of time.

According to preferred embodiments, the holder is extracted to remove residual materials therefrom. For example, in the case of silicone, the holder may include lower molecular weight materials such as unreacted monomeric material and oligomers. The holder may be extracted by placing the holder in an extraction solvent, optionally with agitation. Representative solvents are polar solvents such as isopropanol, heptane, hexane, toluene, tetrahydrofuran (THF), chloroform, supercritical carbon dioxide, and the like, including mixtures thereof. After extraction, the solvent is preferably removed from the holder, such as by evaporation in a nitrogen box, a laminar flow hood or a vacuum oven.

If desired, the holder may be plasma treated, following extraction, in order to increase the wettability of the holder and improve adherence of the drug core to the holder. Such plasma treatment employs oxidation plasma in an atmosphere composed of an oxidizing media such as oxygen or nitrogen containing compounds: ammonia, an aminoalkane, air, water, peroxide, oxygen gas, methanol, acetone, alkylamines, and the like, or appropriate mixtures thereof including inert gases such as argon. Examples of mixed media include oxygen/argon or hydrogen/methanol. Typically, the plasma treatment is conducted in a closed chamber at an electric discharge frequency of 13.56 Mhz, preferably between about 20 to 500 watts at a pressure of about 0.1 to 1.0 torr, preferably for about 10 seconds to about 10 minutes or more, more preferably about 1 to 10 minutes.

A device of the type shown in FIGS. 1 and 2 may be manufactured as follows. The active agent may be provided in the form of a micronized powder, and then mixed with an aqueous solution of the matrix material, in this case PVA, whereby the active agent and PVA agglomerate into larger sized particles. The resulting mixture is then dried to remove some of the moisture, and then milled and sieved to reduce the particle size so that the mixture is more flowable. Optionally, a small amount of inert lubricant, for example, magnesium stearate, may be added to assist in tablet making. This mixture is then formed into a tablet using standard tablet making apparatus, this tablet representing inner drug core 2.

A cylindrical cup of silicone with unitary suture tab 10 is separately formed, for example by molding, having a size generally corresponding to the tablet and a shape as generally shown in FIG. 2. This silicone holder is then extracted with a solvent such as isopropanol. An opening 7 is placed in the silicone holder, for example, with a laser. If desired, a drop of liquid PVA may be placed into the holder through the opening 7 in the holder. Then, the inner drug core tablet is placed into the silicone holder through the same opening 7 and pressed into the cylindrical holder. If the drop of liquid PVA has been applied, the pressing of the tablet causes the liquid PVA to fill the space between the tablet inner core and the silicone holder, thus forming a permeable polymer cap 13 shown in FIGS. 3 and 4. The suture ring is attached to the suture tab by common techniques known in the polymer fabrication industry.

A prior method of making a device of this type includes the following procedures. A cylindrical cup of silicone is separately formed, for example by molding, having a size generally corresponding to the drug core tablet and a shape as generally shown in FIG. 2. This silicone holder is then extracted with a solvent such as isopropanol. Openings 7 are placed in silicone, for example, by boring or with the laser. A drop of liquid PVA is placed into the holder through the open end 13 of the holder. Then, the inner drug core tablet is placed into the silicone holder through the same open end 13 and pressed into the cylindrical holder. As a result, the pressing of the tablet causes the liquid PVA to fill the space between the tablet inner core and the silicone holder, thus forming permeable or semi-permeable layer 5. For the illustrated embodiment, a layer of adhesive (not shown) is applied to the open end of the holder to fully enclose the inner drug core tablet at this end. Tab 10 is inserted at this end of the device. The liquid PVA and adhesive are cured by heating the assembly.

FIG. 3 illustrates another embodiment. In this embodiment, inner drug core 2 may have the form of a tablet, similar to the previous embodiments, including a mixture of active agent 3 and a permeable matrix material 4 such as PVA. A permeable polymer cap 13 is provided to fill the opening 7 of the holder 6.

FIG. 4 illustrates another embodiment of this invention. In this embodiment, inner drug core 2 may have the form of a tablet, similar to the previous embodiments, including a mixture of active agent 3 and a permeable matrix material 4 such as PVA. A permeable polymer cap 13 is provided as a preformed disk which covers the opening 7. In this embodiment, the delivery of the pharmaceutically active agent 3 is controlled by the properties of the permeable polymer cap 13.

It will be appreciated the dimensions of the device can vary with the size of the device, the size of the inner drug core, and the holder that surrounds the core or reservoir. The physical size of the device should be selected so that it does not interfere with physiological functions at the implantation site of the mammalian organism. The targeted disease states, type of mammalian organism, location of administration, and agents or agent administered are among the factors which would affect the desired size of the sustained release drug delivery device. However, because the device is intended for placement in the eye, the device is relatively small in size. Generally, it is preferred that the device, excluding the suture tab, has a maximum height, width and length each no greater than 10 mm, more preferably no greater than 5 mm, and most preferably no greater than 3 mm.

It should be understood that the preferred device comprises a suture tab. However, a suture tab is not necessary for therapeutic operation of the device.

The device is typically provided to the end user in a sealed sterilized package, for example, by gamma irradiation, for example, such as is disclosed in U.S. patent application Ser. No. 10/183,804, the contents of which are incorporated by reference herein.

The examples and illustrated embodiments demonstrate some of the sustained release drug delivery device designs for the present invention. However, it is to be understood that these examples are for illustrative purposes only and do not purport to be wholly definitive as to the conditions and scope. While the invention has been described in connection with various preferred embodiments, numerous variations will be apparent to a person of ordinary skill in the art given the present description, without departing from the spirit of the invention and the scope of the appended claims.

1. A drug delivery device for placement in the eye, comprising:
a drug core comprising a pharmaceutically active agent; and
a holder that holds the drug core, the holder being made of a material impermeable to passage of the active agent and having an opening therein and including a suture tab to aid in securing the device to the eye,
wherein the suture tab contains a suture hole at one end and having a suture ring secured thereto.

2. The device of claim 1, wherein the impermeable material comprises silicone.

3. The device of claim 1, wherein the tab is adhered to at least one of the drug core and the holder.

4. The device of claim 1, wherein the tab is molded integrally with the holder.

5. The device of claim 1, wherein the drug core comprises a mixture of the active agent and a matrix material permeable to said active agent.

6. The device of claim 5, wherein the matrix material comprises polyvinyl alcohol.

7. The device of claim 1, wherein the holder comprises a cylinder that surrounds the drug core, and an end of the cylinder includes the opening.

8. The device of claim 1, wherein the drug core is cylindrical.

9. The device of claim 1, wherein the drug core is coated with a material permeable to said active agent.

10. The device of claim 1, comprising a mixture of pharmaceutically active agents.

11. A package for storing an implantable medical device during storage and shipping, comprising:
an upper surface;
a first flange extending upwardly from the upper surface and defining a containment region for containing the device, said containment region including a support surface for supporting the device in the containment region;
a second flange extending upwardly from the upper surface, said second flange surrounding the first flange and including an upper flange surface for sealing of lidstock thereto; and
at least one side wall extending downwardly from the upper surface and serving to support the package on a work surface,
further comprising a recess extending below the device support surface in the containment region,
wherein the first flange comprises two protrusions extending upwardly from the upper surface and defining the containment region, and the recess has the form of an elongated groove separating the two protrusions and extending transversely to the containment region,
wherein the two protrusions are arcuate, wherein the maximum width between inner surfaces of an individual protrusion is 10 mm, and
wherein the implantable medical device is the device of claim 1.

12. The package of claim 11 wherein the implantable medical device is the device of claim 2.

13. The package of claim 11 wherein the implantable medical device is the device of claim 3.

14. The package of claim 11 wherein the implantable medical device is the device of claim 4.

15. An assembly comprising:
(a) a medical device implantable in the human eye;
(b) a package for storing the device during storage and shipping;
wherein the medical device is the device of claim 1.

16. The assembly of claim 15 wherein the medical device is the device of claim 2.

17. The assembly of claim 15 wherein the medical device is the device of claim 3.

18. The assembly of claim 15 wherein the medical device is the device of claim 4.

19. The assembly of claim 16 wherein the assembly is sterilized.

20. The assembly of claim 16 wherein the assembly is sterilize by gamma irradiation.

21. The assembly of claim 17 wherein the assembly is sterilized.

22. The assembly of claim 17 wherein the assembly is sterilized by gamma irradiation.

23. The assembly of claim 18 wherein the assembly is sterilized.

24. The assembly of claim 18 wherein the assembly is sterilized by gamma irradiation.

25. The assembly of claim 19 wherein the assembly is sterilized.

26. The assembly of claim 19 wherein the assembly is sterilized by gamma irradiation.
